(19) United States
(12) Patent Application Publication
(10) Pub. No.: US 2026/0308661 A1
(43) Pub. Date: Oct. 8, 2026

(54) A Method to Solubilise Polyglutamine Aggregates in Neurological Disease

(71) Applicant: Anthony Brandon Bransgrove, Killara (AU)

(72) Inventor: Anthony Brandon Bransgrove, Killara (AU)

(21) Appl. No.: 19/482,228

(22) PCT Filed: May 10, 2024
(86) PCT No.: PCT/AU2024/050467

§ 371 Date: Nov. 6, 2025

(30) Foreign Application Priority Data
May 11, 2023 (AU) 2023901428

(51) Int. Cl. C12Q 1/6804

(52) U.S. Cl. CPC C12Q 1/6804

(57) ABSTRACT

The present invention relates to a process for the solubilisation and disaggregation of poly glutamine [poly(Q)] peptides using nucleic acids and nucleic acid analogues. The process relies on the discovery that polyadenine will bind to poly(Q) in a specific manner. Which in turn indicates that adenine can bind in a complementary manner to the cis conformed backbone of any peptide or protein through an alignment of their polar groups. The cis backbone structure then, appears able to mimic the part played by uracil or thymine. This is a novel if not revolutionary view of the binding potential of adenine nucleotides and no instance of this mode of binding has previously been identified or communicated.

### TECHNICAL FIELD

The invention relates to the fields of biology and medicine and more particularly to the neurological diseases associated with genetic expansion of CAG repeats, expression of which give rise to expanded polyglutamine (polyQ) tracts housed within a number of different proteins all of which are found as aggregates within the cell. Of these proteins, ten are known to be transcription factors, and each is linked to a distinct neurological disorder, exemplified by Huntington's disease. Isolated native poly(Q) without flanking peptides is highly insoluble as is synthetic poly(Q) in lengths greater than poly(Q15). However poly(Q15) in pure water is soluble for a short time, but will aggregate within 24 hours. The invention is primarily concerned with overcoming the insoluble nature of poly(Q)

### BACKGROUND OF THE INVENTION

The term polyglutamine (polyQ) refers to a stretch of consecutive glutamine amino acids within a protein sequence. These poly(Q) stretches are found in various proteins throughout the body. When the number of consecutive glutamine repeats exceeds a certain threshold, usually 37, it can lead to the formation of abnormal protein folding and the formation of aggregates within the cell body which are associated with neurological disease. Some well known poly(Q) diseases include:
1. Huntington's disease (HD): HD is perhaps the most recognised polyglutamine disease. It is caused by an expanded CAG repeat in the huntingtin gene, leading to the production of a mutant huntingtin protein with an abnormally long poly(Q) tract present in the first exon. This results in a toxic gain of function causing progressive degeneration of neurones, primarily in the basal ganglia and cortex, leading to movement disorders, cognitive decline, assorted psychiatric symptoms and eventually dementia.
2. Spinocerebellar ataxias (SCAs): SCAs are a group of inherited neurological disorders characterised by progressive Ataxia (loss of coordination and balance) together with other neurological symptoms. Several types of SCAs are caused by poly(Q) expansions in different genes, including SCA1, SCA2, SCA3 (Machado-Joseph disease), SCA6, SCA7 and others.
3. Dentatorubral-pallidoluysian atrophy (DRPLA): DRPLA is a rare neurodegenerative disorder caused by expansion of a CAG repeat in the ATN1 gene. It primarily affects the cerebellum, basal ganglia, and cerebral cortex, leading to ataxia, involuntary movements, seizures, and dementia.

The exact process by which expanded poly(Q) tracts lead to neurodegeneration is not known, but protein misfolding and aggregation, coupled with impaired cellular clearance mechanisms and possibly other cellular processes all play a role. Nevertheless the hallmark of these diseases is the intracellular accumulation of aggregates of poly(Q) containing material and a means of facilitating its removal is the purpose of the invention. The invention is concerned particularly with solubilising aggregated poly (Q) and is especially beneficial because it employs nucleic acids or nucleotides as part of the dissolution procedure, and has the advantage of probably reproducing a natural process. Other agents able to bring about solution of pol(Q) aggregates are well known in the art but are mostly toxic and not readily compatible with physiological conditions. Thus trifluoroacetic acid, formic acid, dimethyl sulfoxide, will bring about at least partial solubilisation, of synthetic poly(Q) dependent on the polymer size. Several therapeutic approaches have been explored to combat aggregate formation and mitigate the toxic effects of poly(Q) aggregation in vivo. While no definitive cure currently exists, researchers are investigating various strategies to intervene in the progression of poly(Q) diseases. Some of these approaches include:
1. Small molecule inhibitors can restrain the aggregation of expanded poly (Q) containing proteins, as disclosed in patent number US20180165729A1. These molecules can potentially stabilize the native conformation of the host protein or prevent the formation of toxic oligomers and aggregates. Examples include cystamine, polyphenols, and synthetic peptide inhibitors, but they are ineffective against established aggregates
2. Chaperone based therapy employs proteins able to assist in the correct folding of other proteins preventing misfolding and aggregation. Heat shock proteins (HSPs) are chaperones studied for their potential use against aggregate formation. For instance Hsp70 and Hsp 40 have been shown to modulate the aggregation of expanded poly(Q) containing proteins.
3. Proteasome modulation to enhance degradation of misfolded and aggregated proteins within cells. Enhancing the ubiquitin—proteasome pathway has been attempted. Compounds that activate the proteasome or promote the clearance of misfolded proteins through autophagy have been investigated as disclosed in patent number US20160277467A1
4. RNA interference (RNAi) and anti-sense oligonucleotides against the expression disease related genes or alleles having expanded CAG repeat sequences, as disclosed in patent application number AU 2005201389A1. By selectively targeting the mRNA encoding the disease causing protein, RNAi can potentially reduce expression levels of the toxic protein and halt disease progression. Anti-sense utilizing peptide oligonucleotides as disclosed in patent application number PCT/US2009/051938 is a little more selective but unfortunately this approach reduces wild type protein as well as the mutated form.
5. Immunotherapy is well established to bind with and inactivate extracellular immunogens. In the case of Huntington's poly(Q) disease, and probably other poly(Q) diseases, the pathological protein is abundant in extracellular fluid, cerebrospinal fluid, and plasma, and increasing evidence for the spreading and seeding ability of the protein suggests that immunotherapy would be the ideal solution^{[1]} Intra cellular application of antibody therapy is more complex but utilises ‘intra bodies’ a recombinant antibody fragment delivered to the cell with a viral vector. These therapeutic strategies are all under development and not yet clinically applied.

### Cis-Trans Conformation of Peptides

Poly(Q) tracts are abundant in proteins and have been studied mainly in the context of poly(Q) expansion diseases. Nevertheless more than 4800 human proteins contain these tracts but only 10 of them are associated with disease^{[5]}. Evidently the bulk of poly(Q) insertions within proteins are there to serve a necessary function. The invention herein is concerned particularly with solubilizing aggregated poly(Q), and to this end it has been important to consider its molecular structure in both the soluble and aggregated forms.

Multiple biophysical studies have shown that poly(Q) tracts show different structural behaviour depending on sequence surroundings, consequently different flanking amino acid sequences can give rise to conflicting results^{[14]}. It has been shown that long or short poly(Q) sequences show a predominantly disordered structure in solution and there is also no other obvious difference in their secondary structure. For these studies the simple synthetic polyglutamine of 15 repeats (polyQ15) has been used to minimise issues of context.

This patent application is most relevant to the field of small molecule interventions, potentially able to retard and reverse aggregation of poly(Q). The invention has an advantage over other strategies, in the knowledge that poly(Q) alone or embedded in a host protein is fully conformed as the cis isomer, this unorthodox structure may be responsible for the inability of cellular clearance systems to adequately respond. However with this insight, targeting of poly(Q) has advantages not apparent or available until now.

### SUMMARY OF THE INVENTION

In accordance with the present invention, there is provided a method of dispersing and solubilising poly (Q) and potentially facilitating removal of poly(Q) based, aggregated material found in certain neurodegenerative diseases The present invention had its origins in a study of cell free peptide synthesis and the finding of an inhibitory effect on the process by polyadenine (polyA), that on binding to polyribosomes, attached to a protein and not a nucleic acid. Subsequently a search for this protein was undertaken. Poly(A) is a homopolymer and so it was considered reasonable to look for homopolymer potential binding partners.

In an embodiment of this invention, two of the most commonly encountered homopolymers amino acid repeats, polyalanine and polyglutamine which are frequently associated with genetic expansion disorders, were made up in in stock solutions of 0.1%, and were exposed to 0.01% poly (A): in lengths from about 200 to about 500 nucleotides; or from, about 100 to about 200 nucleotides; or from about 50 to about 100 nucleotides; or from 20 nucleotides to about 100 nucleotides. All of these poly(A) lengths proved to be equally effective in the binding mix however a length of 20 nucleotides (polyA20) with a clearly defined size was the most convenient and has been used throughout the study behind this invention.

Increasing amounts of polyalanine or polyglutamine (15 amino acids each), were exposed to a constant amount of poly(A), and binding between the two was estimated by the magnitude of poly(A) bands migrating on polyacrylamide gel electrophoresis.

FIG. 1 shows a 16% gel segment for the polyalanine binding reaction following electrophoresis and silver staining. All bands are poly (A) running to the anode, they are equivalent in size and intensity with no response to the increasing polyalanine concentrations; The gel does however serve as control comparison for FIG. 2.

The second homopolymer was poly (Q) and significantly a clear response was evident, indicating binding between the two polymers. An incremental gradual loss of the poly (A) band size and intensity occurs as seen in FIG. 2 and this accompanies the stepwise increase of the poly (Q) content. The inverse relationship between the poly (Q) content and the binding reaction band size and intensity does suggest the binding of poly (A) by poly (Q) but a second slower band as seen in a mobility shift assay and attributable to the migrating complex is not apparent. It is not uncommon for a complex to be excluded from the gel, usually because its size is too large for the gel pores,^{[22]} and in the present study this was suspected to be the case because of aggregation of the poly(Q) reactant at the gel surface.

In a further embodiment, an attempt was made to reduce aggregation by using a more soluble form of poly(Q) that had been terminated with two lysine residues poly(Qkk). This failed to reveal a slower second band of the complex, but it did not alter the poly(A) band loss response pattern shown in (FIG. 3).

In another embodiment the robustness of the binding reaction was further tested by using poly(A) when in the form of a double helix. This was an attempt to isolate and protect it from the action of poly(Q). Poly(A) in the binding reaction was replaced with a poly(dA)-poly(dT) duplex, the deoxy form of poly(A) also being a feasible target for the binding assay. FIG. 4 reveals that, unexpectedly the poly (dA)-poly (dT) duplex, employed to contain and shield the poly (dA), appears to be susceptible to the same actions of increasing amounts of poly (Q), showing a similar pattern of band loss to poly (A) although not to completion. The gel also shows a very pale band that increases with intensity along with the increase in poly (Q) content, this band is polythymine (polyT) displaced from the duplex by poly(Q). Thymine is reportedly not stained by silver^{[49]} however, weak staining does occur, and with excess sample it can be recognised. The gel findings indicate there is binding between poly(Q) and poly(dA) and further they show that poly(Q) is energetically able to displace the nonsense strand and open the double helix.

In another embodiment, addition of 10 mM ATP to the same reaction mix poly(Q) as used for poly(A) and qualitatively separating the origin material from that migrating on cellulose TLC with a water solvent, showed complete dissolution with migration of aggregated material normally found lodged at the origin. The suggestion being that ATP is also able to bind poly(Q) and solubilise it.

A further embodiment using this simple system but with 0.1% formamide replacing ATP showed a similar result with poly(Q).

### Stereochemical Modelling

Binding of poly(A) to poly(Q) is likely to have a structural basis that can be identified at the molecular level.

In an embodiment of this invention preliminary modelling with an adenine nucleotide construct, attempted to engage with a generic peptide backbone structure in the form of a tripeptide. This failed to reveal any suitable backbone polar group alignments with adenine which might explain the binding between poly(A) and poly(Q). Nevertheless, it was considered important to examine all aspects of the physical geometry of the peptide, and with this in mind, the orthodox peptide bond in the trans conformation was rotated to the cis form. Significantly, this conformation of the same peptide did show polar group alignments appropriate for H-bond formation between the peptide backbone and adenine.

In the trans conformation, successive side chains are positioned on opposite sides of the peptide backbone and are directed away from each other, similarly for the polar groups, which alternate between sides. In the cis conformation, successive side chains are positioned on one side of the backbone with the same directionality, while polar groups are positioned together in pairs, carbonyl and amine, on the opposite side.

The assembly of side chains to the one side of the backbone with a cis conformation presents a unique opportunity to explore side-chain-side-chain interactions not previously entertained with trans conformers. In particular, aromatic or polar side chains might be expected to interact intimately with one another^{[23]} in a manner not sterically possible with the trans peptide. For the present invention this observation was a lead in to understand the natural default mechanism underlying the formation of polyglutamine as the cis conformation.

### Energy Barriers for ω Torsion

The negative gel results obtained for poly(A) exposure to alanine peptides is not surprising because conversion of the trans peptide into the energetically less favourable cis conformer is governed by the amide partial double bond with a rotational barrier of ~20 kcal mol−1.^{[24]} Equally the energy barrier from cis to trans is of the same order of magnitude at ~18 kcal mol−1.^{[24]} Adenine base pairing with uracil or thymine has been variously estimated at 12.8-18 kcal mol−1.^{[24]} A similar energy might be expected for adenine pairing with the cis peptide, but this would be insufficient to overcome the @ rotational barrier and flip from trans to cis. Consequently, in the absence of suitable isomerases or other energy inputs, poly(A) would be restricted to binding (if at all) only to pre-existing cis-peptide chains The invention is founded on the probability that a pre-existing cis peptide is the normal conformation adopted by poly(Q).

### Adenine: Cis Peptide Construct

The complex between poly(Q) and adenine, its analogues and congeners, is the most critical component of the invention.

An embodiment of this criticality is possible because both the peptide group and adenine base are planar, it is possible to show a two-dimensional schematic of their interaction in some detail (FIG. 5a, b). The atomic coordinates cited are taken from Pauling^{[4]} with minor refinements to adenine from Voet and Rich.^{[26]} On the left side, in FIG. 5a is shown the peptide backbone in the trans conformation (ω=180°). It appears to lack any suitable polar group pair in an appropriate position or orientation to engage with adenine, confirming preliminary findings with molecular models. On the right side (FIG. 5b) the last peptide bond has been rotated through 180° to the cis conformation (ω=0°) this brings about several changes to the peptide. As mentioned above, the side chains are on the same side of the backbone, and the polar groups are positioned together on the opposite side, now with similar directional orientation but diverging a little from each other.

These changes are relevant because they appear to allow possible H-bond interactions with adenine (FIG. 5b): the backbone carbonyl oxygen with adenine N6 amine and the backbone amine with the adenine ring nitrogen N1. The bond to the oxygen targets one lone pair, situated to the side, as also occurs with a nucleic acid base pair. H-bond distances are set at ~2.9 Å, or a little less than the sum of the van der Waals radii, both H-bond angles appear to be close to 180° with donor, hydrogen and acceptor all in a straight line maximising the bond strength. Importantly, like the trans isomer, the cis isomer is planar, the base is planar and the H bonds unite the two giving a coplanar structure (FIG. 5b).

The cis peptide appears to be complementary to adenine in the same fashion as is the base uracil. A comparison of the cis peptide and uracil suggests that the relevant bond lengths and angles are broadly similar and so they might well be interchangeable (FIG. 6). The schematic suggests an adenine monomer can pair with and potentially bind the cis dipeptide but it doesn't indicate whether the adenine polymer can bind to an all-cis peptide polymer. This is an important consideration because successive polar groups along the two chains must be in register and alignment to generate a stable and functional interaction between the two polymers.

### Interaction of poly(Q) and poly(A)

In a further embodiment a construct of an all-cis poly(Q) was assembled independently of poly(A). It was then used as a scaffold against which a poly(A) chain was positioned and polar group alignment could be assessed for potential bond formation as described above for the monomeric adenine.

Modelling of a hexapeptide of glutamine in the all-cis conformation revealed a helical and potentially flexible structure, as previously predicted.^{[27]} in which adjacent side chain carboxamide groups are close enough to permit H-bond formation. These multiple inter-amide side chain links are able to extend the full length of the peptide forming a second chain parallel to the cis backbone. The second chain appears to play a significant role in fixing and stabilising the geometry of the poly(Q) helix, particularly because it is able to exploit the cooperatively augmented H-bond strength (as outlined below).

Stereochemically, the construct indicates that the backbone of this cis poly(Q) structure forms a regular right hand helix ~6.4-Å diameter with a pitch of ~28 Å and 9-10 residues per turn. The omega torsion angle is 0°, phi and psi torsion angles are close to −150 and +150° respectively, placing them in the beta zone of a Ramachandran and Mitra plot.^{[19]} The backbone polar groups NH and CO are paired and directed radially from the exterior of the helix, and the whole appears to be free from van der Waals contacts. The carboxamide H-bonded chain is ~10.5 Å in diameter and its augmented co-operative H-bond strength might play a role in constraining the backbone as the cis isomer, as well as setting other parameters of the helix. The rotamer angles positioning the donor and acceptor groups at 2.8-2.9 Å apart (implying bonding) are: chi 1 (Cα−Cβ)=+10°, chi 2 (Cβ−Cγ)=180° and chi 3 (Cγ−Cδ)=+65°. Cδ is coplanar with the planar carboxamide group.

Single stranded poly(A) is part helical and part random coil,^{[28]} with freedom of rotation around bonds of the sugar phosphate backbone and the glycosidic bond. This essential flexibility allows the helical configuration of poly (A) to pair its base polar groups with those of the cis peptide backbone recurrently and extending for the length of the two bound chains. As such, their respective orientation is: poly(Q) is NH→CO to poly(A) 5′→3′. The plane of the peptide bond in the poly(Q) helix is ~30° to the axis, which means for coplanarity with adenine, the base must roll to the same plane by rotation around the glycosidic bond, which is appropriately oriented with ribose almost perpendicular the base. The poly(A) helix is right handed and coaxial with poly(Q), which it surrounds. Its diameter is 23.5 Å with 9.5 residues per turn. The pitch is 28.0 Å with a residue rise of almost 3.0 Å. This resembles a single strand of the A form of DNA,^{[29]} with a larger diameter and a shorter pitch than the B form, also a base inclined to the axis and a central void, which in the present proposal is occupied by the poly(Q) helix. It differs from the A form with a lower number of bases in a full turn (9.5 v11).

FIG. 7 shows a schematic projection of the complex normal to the common axis. The sugar phosphate backbone is represented by the heavy circle. Internal to this is the base, then the cis peptide, both represented in part as blank plates or plaques^{[30]} to reduce the information and aid clarity of understanding the structure.^{[31]} Molecular details are indicated in one quadrant. The position of the side chain carboxamides in an H-bonded train is shown, except for the quadrant with molecular detail, and the coplanar base and peptide group are represented on an oblique plane. The stability of this complex appears to rely on the number of inter-carboxamide H bonds that can be formed, and these are set by the poly(Q) chain length. However it has been found that a poly(Q) chain length of less than 10 units does not aggregate in the usual manner, suggesting a structural change, possibly a conformation, has collapsed.^{[31]} The ability of poly(A) to bind poly(Q) might not be a feasible outcome therapeutically for the invention, because of molecular size, however it does set the theme that small molecules such as adenine based monomers might be preferred.

### ATP Action on Poly(Q)

ATP has been known for a long time to play some part in maintaining the solubility of cellular proteins.^{[50]} Initially considered to be an energy dependent process, able to maintain proteostasis by acting on cellular chaperones such as the Hsp70 complex, to correctly fold proteins and possibly reverse aggregation.^{[51]} More recently a second function of ATP has been proposed, that relies on its ability to act as a hydrotrope^{[52]} able to stabilise and suppress protein thermal instability or aggregation [PA. 1305-5403-MSG-ZE] Hydrotropes are amphiphilic molecules able to solubilise both hydrophilic and hydrophobic molecules, they differ from surfactants in requiring far higher concentrations to be active, in the case of ATP 4-10 mM. This might well explain the high levels of cellular ATP well above those needed for metabolic hydrolysis energy purposes.

The studies supporting this application are also concerned in a broad sense with stabilizing or solubilising proteins but they differ clearly from utilising the hydrotropic effects of ATP because they are centred on the finding that adenine based molecules can bind specifically to poly(Q) and that this binding is because the peptide backbone has assumed a cis conformation. Cis peptide backbones other than of poly(Q) would also likely fit the requirements for binding. It is also possible that some effects attributed to ATP hydrotropic function may in fact be a function of its binding to a cis conformation of a peptide backbone

This invention is founded on the hypothetical proposition that poly(Q) exists with a cis conformation of its backbone. There are five arguments in support of this proposal and reiterated at this point.
1. The gel electrophoresis evidence of binding between poly(A) and poly(Q); and this depends on complementary binding between a cis backbone and adenine.
2. The biophysical studies showing clearly that all cis peptide helices are possible and likely, and because of energy barriers, once formed they are likely to be stable.^{[24, 27,30]}
3. Again, biophysical studies of formamide crystals explicitly describe the default molecular structure of maximally H bonded formamide.^{(2,3)} The parallel possibilities for glutamine side chain amides are obvious.
4. Poly(Q) monomers longer than about 12 residues undergo a physical collapse^{[20]} which is associated with diminished water solubility.^{[14]} The poor water solubility would likely result from withdrawal of amide polar groups from the surrounding water to preferentially bind with amides of adjacent glutamine side chains within the molecule and probably is the basis of the cis isomer structure.
5. Poly(Q) expansion diseases have defective clearance mechanisms for misfolded proteins. Many of the flanking peptides or other aggregated proteins are eventually processed but leave behind the poly(Q) tract which is resistant to attack by most known proteases. It seems the cis backbone structure does not satisfy the physical requirements of proteases and must isomerise to trans before digestion.^{[40]} Thus protease resistance may indicate cis.

### BRIEF DESCRIPTION OF THE DRAWINGS

FIG. 1. Gel image of binding reaction between poly (Alanine) and poly (A), showing the same poly(A) band intensity for all reaction mixtures containing increasing poly(Alanine) amounts. The grey wedge represents the peptide concentration for each of the gels.

FIG. 2. Gel image of binding reaction between poly(Q) and poly(A), showing progressive loss of band intensity proportional to the binding mix poly(Q) content. No second slower group of bands is observed to indicate a complex is formed.

FIG. 3. Gel image of binding reaction between poly(QKK) and poly(A). Despite solubility enhancement the loss of band intensity is clearly apparent and similar to that seen using poly(Q) in FIG. 2.

FIG. 4. Gel image of the binding reaction between the poly(dA)-poly(dT) duplex and poly(Q). There is a progressive loss of poly(dA)-poly(dT) duplex band intensity with increasing poly(Q) content.

FIG. 5. Schematic showing peptide backbone polar group alignments with those of adenine for trans (a) and cis (b) peptides.

FIG. 6. A comparison of a cis backbone peptide structure, with that of uracil.

FIG. 7 Schematic showing relative coaxial positions and geometry of helical poly(Q10) encircled by helical poly(A10). The glutamine side chain carboxamide groups lie between the base and the peptide backbone. Potential H bonds between the two helices are indicated by broken lines. Viewing is down the axis.

FIG. 8. (a) Crystal structure of formamide with cooperatively augmented H bond distances of 2.88 A (lengthwise chain) and cross-linking H bonds distance of 2.93 (b) Likely structure of the poly(Q) dimer cross-linked to the formamide chain to help illustrate bonding extent and potential to form kinked planar sheets, and showing Cγ as part of the plane but continuous with the glutamine side chain and the cis conformed backbone.^{[2,3]} Based on schematic FIG. 1 in ref.^{[3]}

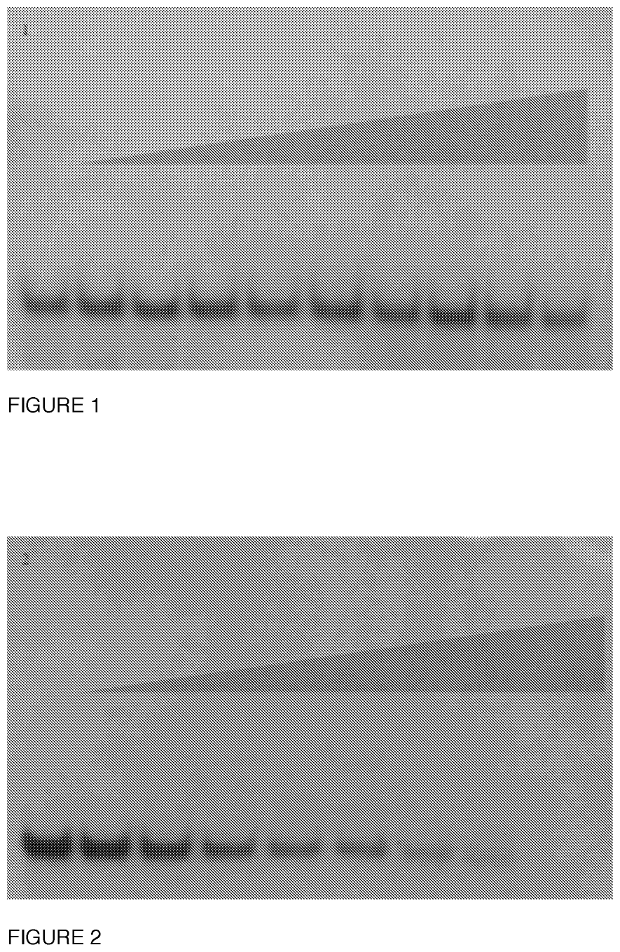

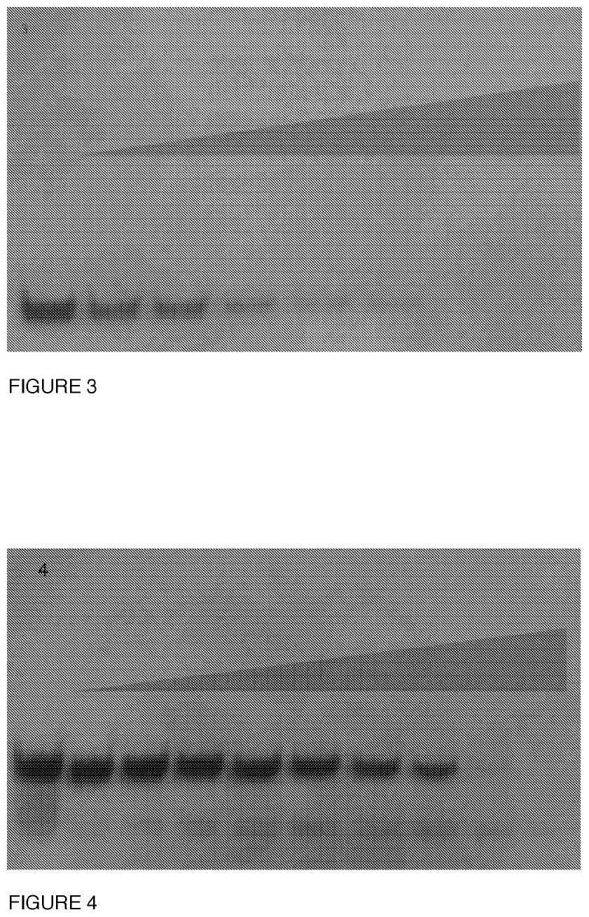

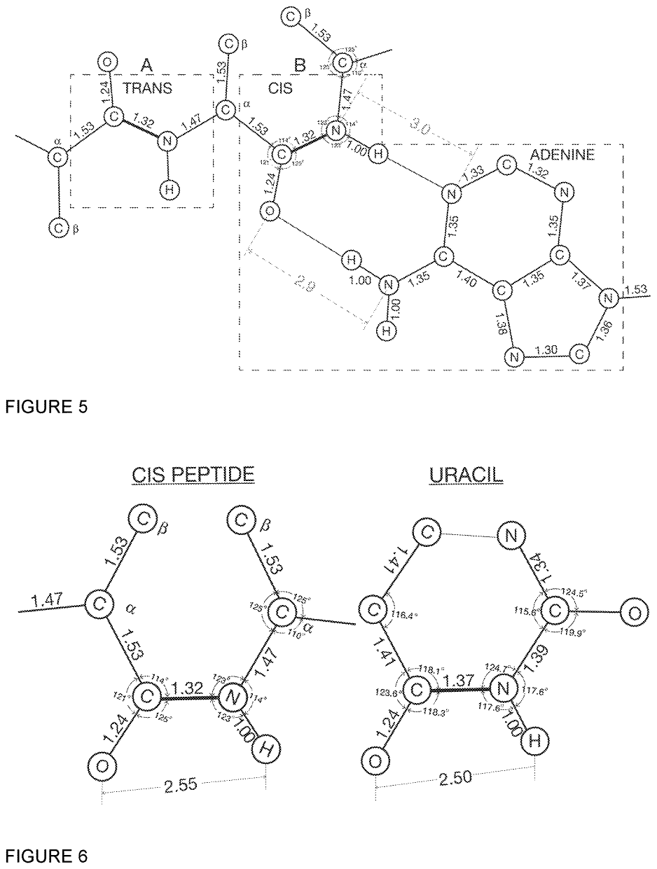

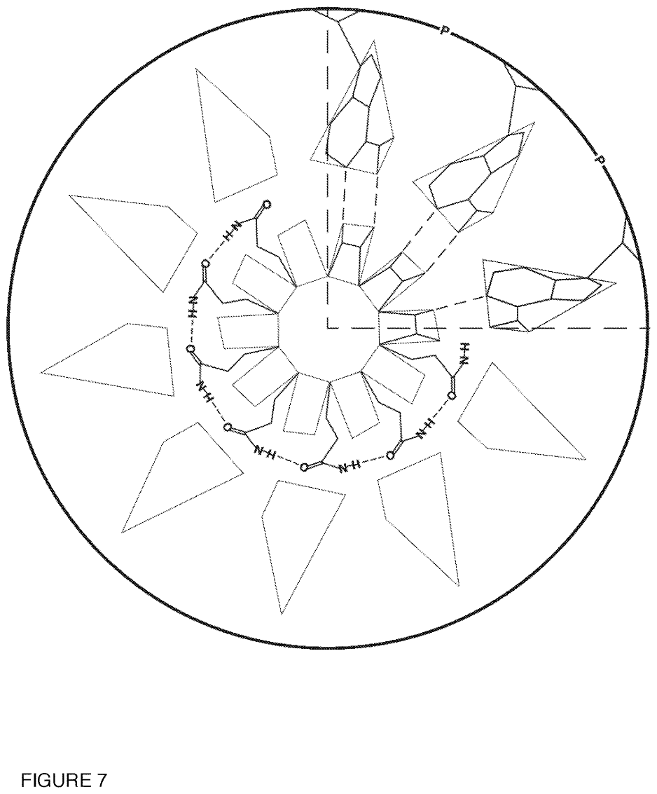

### DETAILED DESCRIPTION OF EMBODIMENTS

Gel electrophoresis indicates poly(A) binds to poly(Q). Stereochemical modelling suggests adenine is able to H bond to the all cis conformation of the generic peptide backbone, rather than requiring a particular amino acid polymer. Modelling further indicates that the mode of poly(A) binding to poly(Q) is by formation of a coaxial helix surrounding the smaller central poly(Q) helix. This complex would likely stabilize the poly(Q) as the monomer, making it unavailable as a target for binding with more poly(Q). Free poly(Q) then is reluctant to exist as a monomer, but instead rather as dimers or oligomers. This state takes the form of insoluble aggregates or soluble aggregates which progress to the insoluble form usually within hours. The poly(Q) used experimentally for this invention was a polymer of 15 glutamines with no added charged amino acids to aid solubility (except where described above). It dissolved in pure water but aggregated within 24-48 hours, it also aggregated quickly on addition of buffer. The structure of aggregates is further described below. The substance of this patent application is a suggested means for dissolving aggregated poly(Q), the need for this arises because of its the frequent occurrence in and likely causation of, many inherited neurodegenerative diseases.

Peptide homopolymers and their origin: Microsatellite DNA, also known as simple sequence repeats (SSRs) are tracts of 1-6 base pairs, repeated typically 5 to 50 times^{[6]}. Of these simple sequences, trinucleotide repeats are codon length and when present in coding regions specify homopolymer tracts of amino acids carried within the host protein. These amino acid repeats are found in a large number of eukaryotic and prokaryotic proteins including approximately 20% of human proteins^{[7]} suggesting they serve a normal and necessary functional role.

Mutability: Microsatellites are highly mutable^{[8]} particularly in many cancers for which instability giving rise to expansions can be both predictive and prognostic for the disease^{[9]}. In non-malignant disorders expansions of these tracts can become pathological when a threshold size is reached^{[10,11]} and they are associated with inherited diseases particularly neurodegenerative states where intracellular aggregates of the homopolymer along with its harbouring protein appear. It has recently been reported that microsatellite expansions are responsible for more than 50 human genetic diseases.^{[12]}

Expansion disorders: Prominent amongst these are the CAG repeats coding for glutamine, GCC repeats, coding for alanine and CGG repeats, coding for glycine. There are 10 known polyglutamine expansion disorders, exemplified by Huntington's disease (HD). All have a pathological length threshold of about 37 repeats,^{[10,13]} all are within transcription factors and all are neurodegenerative diseases.^{[14]} Huntington's Disease (HD) is an autosomal dominant inherited disorder with an incidence of 5-10 per 100,000 individuals in Europe and North America (Walker, 2007). HD is caused by the expansion of CAG trinucleotide repeats within the first exon of the huntingtin (HTT) gene, leading to disruption of protein function, and neurodegeneration. In the case of polyalanine there are nine known expansion disorders with a pathological length threshold of ~20 repeats.^{[15]} Of these, eight involve developmentally important transcription factors and are responsible for causing congenital malformations.^{[16]} Polyglycine expansion disorders exceed five and have a pathological length threshold of ~50 repeats.^{[12]} They are noteworthy because they frequently arise by non-canonical replication from non-coding gene regions.^{[17]} Foremost within this group are Fragile X mental retardation (FXMR) and Fragile X tremor with ataxia (FXTAS).^{[18]}

The peptide bond: The peptide bond has a partial double bond character that restricts free rotation around the bond to two torsion angles of ~0 and 180°, causing the peptide group of six surrounding atoms to all lie in the one plane. Peptide bonds are mostly found in the trans configuration with a torsion angle ω close to 180°; however, a small number in the non-proline cis conformation also occur,^{[19]} with ω angles close to 0°. But these are extremely rare, about ~0.05%,^{[30]} and this refers to single peptides only, not repetitive all-cis tracts. This scarcity, together with misconceptions about adjacent side chain clashes^{[24]} (only possible if phi and psi torsion angles were not free to rotate), and the energetic cost of ~2 kcal mol-1 to stabilise the cis over the trans conformation, are the three main reasons^{[27]} why the cis peptide conformation has been mostly considered irrelevant.

Rarity of the cis backbone: The scarcity, however, may be more apparent than real; it has been suggested recently in a review of the Protein Data Bank using an unambiguous assessment method that almost 5000 cis peptide bonds have been incorrectly assigned as trans.^{[32]} If poly(Q) tracts in proteins are in the default cis conformation but unrecognisable as such because they are within intrinsically disordered domains, or because of the aggregation prone nature of poly(Q), then a relatively large cohort of cis peptides may exist and be part of the above misassigned group.

### Evidence for Cis Backbone Conformations

Apart from the gel electrophoresis indication of binding between the two polymers and the explanation for this by structural assignment of cis backbone polar groups complementing those of adenine, there are other indicators of the truth of this assumption.

Aggregate structure: An important outcome of this study, in the context of a main-chain cis conformation of poly(Q), has been the probability of H bonding between carboxamide groups of adjacent side chains which may link them together over the length of the cis peptide. Side-chain side-chain preferred bonding sets them apart from main-chain polar groups and would reduce the possibility of interaction between the two. In support of this notion are reports emphasising selective intrastrand side-chain-side-chain bonding, rather than inter-strand.^{[33-35]} This structure would be compatible with the cis isomers only if it exclusively involved adjacent glutamine side chains.

### Formamide and Cooperativity Energy Levels:

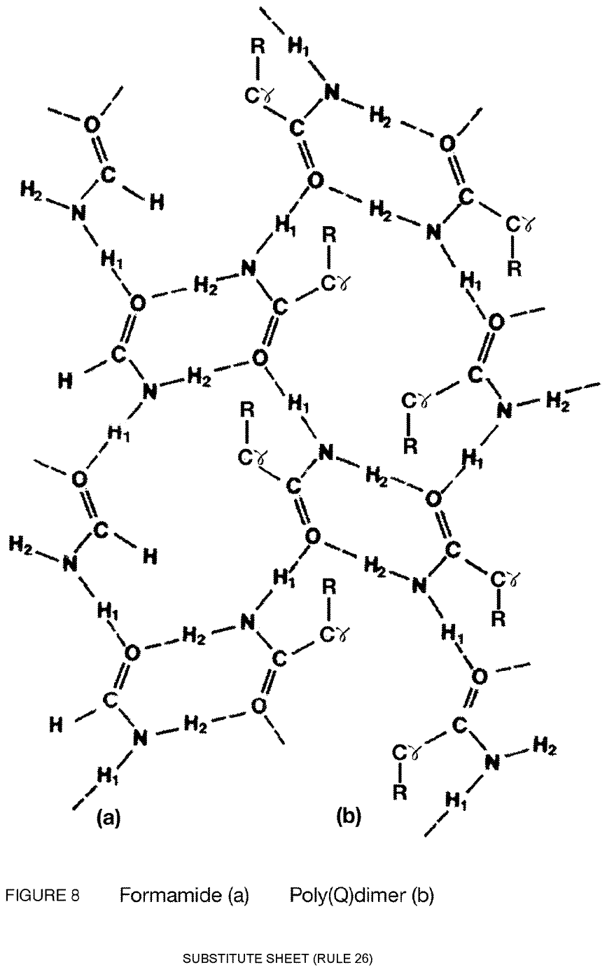

H-bonded chains of formamide closely resemble those found in proteins, and demonstrate that cooperativity is the main driving force for preferred intrastrand bonding (FIG. 8a).

Importantly, formamide derived data show that linear extension of an H-bonded amide chain has a cooperative effect,^{[35]} giving an energetic enhancement for each additional bond.

Depending on the chain length, the H-bond strength can increase by greater than 200%,^{[36]} and in proteins this sets the stage for a selection process giving rise to inter-side chain amide H bonding in preference to bonding with multiple other polar groups in the vicinity, particularly those of the peptide backbone. The energies of individual bonds in a chain depend on the chain length and position of the bond in the chain.^{[37]} Experimental data for the decamer chain of formamide^{[36]} show the central H-bond strength as 12.87 kcal mol-1, which is ~2.5×the single H bond of 4.49 kcal mol-1 in the dimer.^{[37]} Bond strength enhancement of this magnitude would be sufficient to selectively build polycarboxamide links between each side chain establishing a continuity between each glutamine residue in addition to the peptide backbone.^{[3]} But even a 250% bond strength enhancement will be less than the 20 kcal mol-1 energy barrier needed to be overcome to flip the trans amide to cis. Nevertheless, the fixed and proximate position of the polar groups of poly(Q) (not available to freely diffusing formamide), and chains longer than 10 residues, such as 15 residues of the invention could provide the added impetus to breach that energy barrier, suggesting that cis poly(Q) might well self assemble. Wild type poly(Q) containing proteins are polymorphic with a range of 10 to 37 residues, implying the lower limit for self assembled cis formation may be ~10 residues. The presence of an H bonded carboxamide chain running the length of the poly(Q) would ensure the backbone is cis conformation, but how to confirm its presence?

Monomer replication: Further formamide derived data show the chains and 6 atoms of the amide bond, including side chain Cγ atom are planar and give rise to puckered sheets.⁽²⁾ The H bond augmented poly(Q) monomer chain can potentially function as a scaffold onto which further monomer chains can be bind by H bond cross links to give multiple layers of carboxamide side chains of poly(Q) (FIG. 8b). In the case of the dimer the chains are antiparallel, so permitting potential H bond interaction between their respective cis conformed backbones. Out of register multiple monomer additions to the scaffold poly(Q) might well provide a mechanism for extension of a growing fibril, while layers of cross-linked monomers would generate extended sheets normal to the fibril axis.

The ability of poly(Q) side chain amides to interact not just for the augmented chain links but also to maximally utilise the total of 4 H bonds of each carboxamide group by the further addition of crossed linked monomers, means the polar structure is consumed and not available for H bonding with the solvent water. The failure to interact with water retards the expansion potential of the polymer and it remains in a compact state.

Finally soluble poly(Q) monomers or aggregates form an unusual structure in water^{[14]} with a loss of intrinsic viscosity not expected with for a hydrophilic molecule, it loses the bulk of its interaction with water.^{[21]} This state is described as a structural collapse^{[20]} and it occurs with chain lengths greater than about 10-12 units. The collapse seems to be associated with the formation of internal H bonds, very possible causally related to a backbone flip from trans to cis.

Protease insensitivity: Poly(Q) expansion disorders generate cellular aggregates composed of host protein and other proteins along with the embedded poly(Q) tract. These components resist breakdown by both the ubiquitin proteasome system and the autophagy and lysosomal system,^{[38]} however, the normal protein components are processed leaving behind poly(Q) tracts that resist degradation.^{[39]} Proline peptides have a high proportion of bonds as the cis isomer, and this has served as a test system for the protease sensitivity of cis peptides. It has been reported that exopeptidases, prolidase and aminopeptidase P, are unable to hydrolyse the cis isomer but instead are specific for the trans isomer of the X-L-proline peptide bond. The cis form must isomerise to the trans before it can be hydrolysed.^{[40]} Prospective studies need to determine if the trans peptide bond is a general requirement for proteolytic activity, and if so, it might explain why the poly(Q) peptide as a cis isomer is resistant to, if not unaffected by, proteolysis.^{[39]}

Nascent peptide: Returning to the function of poly(A) as the tail of mRNA in vivo, it is of course in close proximity to the ribosome with its protein synthesising machinery. All surrounding proteins on or off the ribosome would be established as secondary or tertiary structures based on a trans backbone. With respect to a cis backbone able to bind with the poly(A) tail, the only candidate likely to fill the role would be the unfolded nascent peptide as it exits the ribosome tunnel. Peptides are synthesised at the peptidyl transferase centre (PTC) of the ribosome by successive addition of amino acyl tRNA residues to the c-terminus of the nascent chain. If each new amino acyl tRNA enters the A site of the PTC with the same orientation, then newly synthesised peptide bonds at the P site should all be stereochemically identical, suggesting de novo origin of all cis bonds with no requirement for energy expenditure creating the alternating bonds of the trans isomer. Because of the 14 kcal mol-1 or more energy barrier for the cis into trans conversion, it is likely that the cis peptide isomer once formed in the PTC would be sufficiently stable to negotiate the full length of the exit tunnel. The all-cis peptide has extended backbone torsion angles placing it in the B zone of the Ramachandran and Mitra plot,^{[19]} suggesting that it can form either a B strand or, as with poly(Q), an extended helix (structurally not greatly dissimilar). There are many reports describing peptide secondary structure within the ribosome exit tunnel, mostly of compaction, thought to be probable alpha helices^{[41]} but also of B strands.^{[42]} It seems that helices can form when close to the PTC but change to B structures on movement towards the exit.^{[42]} Moreover, it has been suggested that all nascent peptides, regardless of sequence, emerge from the tunnel with the same uniform B-strand like conformation,^{[43]} which equates to extended backbone torsion angles resembling a cis conformation. The poly(A) tail is in the immediate vicinity of the lead ribosome (of a polysome) with its emerging (possibly cis) nascent peptide a possible binding partner for the poly(A) tail. Binding to the nascent peptide backbone would very likely affect the translation process—speeding, slowing or halting it. In addition, it might assist in the process of peptide folding leading eventually to a trans conformation and disengagement from the poly(A) tail, in turn voiding its effect on the translation process.

Transcription factors (TFs): Apart from the mRNA tail, other poly(A) and poly(dA) sites exist within the cell, particularly in the genetic regulatory regions of promoter and enhancer of most organisms at the 5′ end of the gene.^{[44]} These are short, usually a run of 4-6 bases^{[45]} and flanked by alternating thymine residues as seen in the native TATA box sequence, which is one of several distinct elements of the core promoter responsible for assembling the transcription complex with RNA polymerase II. An oligomer TATATAAAA is known to adopt the DNA A form structure,^{[29]} which interestingly is similar to the conformation of the poly(A) of the proposed complex with poly(Q). Promoters are targeted by TFs, which, on binding, activate the transcription process. TFs appear to consist of a DNA binding domain (DBD) and one or more intrinsically disordered regions (IDRs).^{[46]} Many TFs contain poly(Q) tracts^{[47]} that appear to be part of the IDRs and have been shown to stimulate transcription.^{[48]} The 10 known poly(Q) neurodegenerative disorders all have TFs carrying expansions of their normal poly(Q) complement. However, normal length poly(Q) tracts in a TF may be suitably positioned to bind with the multiple adenine residues present in promoters. The finding in this study that poly(Q) is able to bind with poly(dA) when formed in a double helix, by displacing poly(dT), suggests this as a possible mechanism TFs might employ to open a double helical section of the promoter. But why would poly(A) bind to poly(Q) in preference to binding with poly(T)? Duplex oligonucleotides are prone to separate because of the mutual repulsion of their backbone charged phosphate groups, often able to be masked with salt solutions. In the case of poly(Q), which is electrically neutral, a complex with poly(A) would be devoid of the repulsion element and so allow its preferential binding.

Polyglutamine when greater than about 8-10 residues in length, is susceptible to collapse and a change of its backbone conformation from trans to cis. This process relies on the creation of a second chain of H bonded amide groups extending the length of the polymer in parallel with the first, which is the peptide backbone or main-chain. The two chains holding the poly(Q) together are sensitive to different influences the cis backbone to nucleotides or adenine, while the H bonded inter-side chain amides would require H bond breaking with like substances, in particular formamide.

### References

1] Denis H L., David L S., Cicchetti F. Antibody based therapies for Huntington's disease: current status and future directions. Neurobiology of Disease. 2019: 132: doi.org/19.1016/j.nbd.2019.1045569
[2] Ladell J., Post B. The Crystal Structure of Formamide. Acta Cryst. 1954: 7: 559-564.
[3 Bellissent Funel M C., Nasr S., Bosio L. X-ray and neutron scattering studies of the temperature and pressure dependence of the structure of liquid formamide. J. Chem. Phys. 1997: 106: 7913-7919
[4] Pauling L. The Nature of the Chemical Bond, 3rd edn. Cornell University Press; 1960.
[5 Mier P., Andrade-Navarro M. Between interactions and aggregates: The poly(Q) balance. Genome Biol. Evol. 2021 doi:10.1093/gbe/evab246
[6] Tautz D, Renz M. Simple sequences are ubiquitous repetitive components of eukaryotic genomes. Nucleic Acids Res 1984; 12: 4127-4138. doi:10.1093/nar/12.10.4127
[7] Mularoni L, Ledda A, Toll-Riera M, Albà M M. Natural selection drives the accumulation of amino acid tandem repeats in human proteins. Genome Res 2010; 20: 745-754. doi:10.1101/gr. 101261.109
[8] Darling A L, Uversky V N. Intrinsic disorder in proteins with pathogenic repeat expansions. Molecules 2017; 22: 2027. doi:10.3390/molecules22122027
[9] Amato M, Franco R, Facchini G, Addeo R, Ciardiello F, Berretta M, Vita G, Sgambato A, Pignata S, Caraglia M, Accardo M, Zito Marino F. Microsatellite instability: from the implementation of the detection to a prognostic and predictive role in cancers. Int J Mol Sci 2022; 23: 8726. doi:10.3390/ijms23158726
[10] Adegbuyiro A, Sedighi F, Pilkington IV A W, Groover S, Legleiter J. Proteins containing expanded polyglutamine tracts and neurodegenerative disease. Biochemistry 2017; 56: 1199-1217. doi:10.1021/acs.biochem.6b00936
11] Perutz M F, Johnson T, Suzuki M, Finch J T. Glutamine repeats as polar zippers: their possible role in inherited neurodegenerative diseases. Proc Nat Acad Sci USA 1994; 91: 5355-5358. doi:10.1073/pnas.91.12.5355
[12] Boivin M, Charlet-Berguerand N. Trinucleotide CGG repeat diseases: an expanding field of polyglycine proteins. Front Genet 2022; 13: 843014. doi:10.3389/fgene.2022.843014
[13] Wen J, Scoles D R, Facelli J C. Molecular dynamics analysis of the aggregation propensity of polyglutamine segments. PLoS One 2017; 12: e0178333. doi:10.1371/journal.pone.0178333
[14] Wetzel R. Physical chemistry of polyglutamine: intriguing tales of a monotonous sequence. J Mol Biol 2012; 421: 466-490. doi:10.1016/j.jmb.2012.01.030
[15] Oma Y, Kino Y, Toriumi K, Sasagawa N, Ishiura S. Interactions between homopolymeric amino acids (HPAAs). Protein Sci 2007; 16: 2195-2204. doi:10.1110/ps.072955307
[16] Li L, Ng N K L, Koon A C, Chan H Y E. Expanded polyalanine tracts function as nuclear export signals and promote protein mislocalization via eEF1A1factor. J Biol Chem 2017; 292: 5784-5800. doi:10.1074/jbc.M116.763599
[17] Boivin M, Deng J, Pfister V, Grandgirard E, Oulad-Abdelghani M, Morlet B, Ruffenach F, Negroni L, Koebel P, Jacob H, Riet F, Dijkstra A A, McFadden K, Clayton WA, Hong D, Miyahara H, Iwasaki Y, Sone J, Wang Z, Charlet-Berguerand N. Translation of GGC repeat expansions into a toxic polyglycine protein in NIID defines a novel class of human genetic disorders: the polyG diseases. Neuron 2021; 109 1825-1835.e5. doi:10.1016/j.neuron.2021.03.038
[18] Liufu T, Zheng Y, Yu J, Yuan Y, Wang Z, Deng J, Hong D. The polyG diseases: a new disease entity. Acta Neuropathol Commun 2022; 10: 79. doi:10.1186/s40478-022-01383-y
[19] Ramachandran G N, Mitra A K. An explanation for the rare occurrence of cis peptide units in proteins and polypeptides. J Mol Biol 1976; 107: 85-92. doi:10.1016/S0022-2836(76)80019-8
[20] Dougan L, Li J, Badilla C L, Berne B J, Fernandez J M. Single homopolypetide chains collapse into mechanically rigid conformations. Pro Natl Acad Sci 2009: 106: 12605-12610
[21] Feng M, Bell D, Wang Z, Wei Z. Length dependent structural transfere of Huntingtin poly(Q) domain upon binding to 2D nanomaterials. Front. Chem. 2020 8: 299.doi. org/10.3389/fchem2020.00299
[22] Seo M, Lei L, Egli M. Label-free electrophoretic mobility shift assay (EMSA) for measuring dissociation constants of protein-RNA complexes. Curr Protoc Nucl Acid Chem 2019; 76: e70. doi:10.1002/cpnc.70
[23] Jabs A, Weiss M S, Hilgenfeld R. Non-proline cis peptide bonds in proteins. J Mol Biol 1999; 286: 291-304. doi:10.1006/jmbi. 1998.2459
[24] Mathieu S, Poteau R, Trinquier G. Estimating the “steric clash” at cis peptide bonds. J Phys Chem B 2008; 112: 7894-7902. doi:10.1021/jp711082d
[25] Jurecka P, Hobza P. True Stabilization energies for the optimal planar hydrogen-bonded and stacked structures of guanine . . . cytosine, adenine . . . thymine, and their 9- and 1-methyl derivatives: complete basis set calculations at the MP2 and CCSD(T) levels and comparison with experiment. J Am Chem Soc 2003; 125: 15608-15613. doi:10.1021/ja036611j
[26] Voet D, Rich A. The crystal structures of purines, pyrimidines and their intermolecular complexes. Prog Nucleic Acid Res Mol Biol 1970; 10: 183-265. doi:10.1016/S0079-6603(08)60565-6
[27] Poteau R, Trinquier G. All-cis helical polypeptides. J Org Chem 2007; 72: 8251-8258. doi:10.1021/jo0711790
[28] Saenger W, Riecke J, Suck D. A structural model for the polyadenylic acid single helix. J Mol Biol 1975; 93: 529-534. doi:10.1016/0022-2836(75)90244-2
[29] Dickerson R E, Ng H-L. DNA structure from A to B. Proc Natl Acad Sci USA 2001; 98: 6986-6988. doi:10.1073/pnas.141238898
[30] Poteau R, Trinquier G. All-cis cyclic peptides. J Am Chem Soc 2005; 127: 13875-13889. doi:10.1021/ja052342g
[31] Perutz M F, Finch J T, Berriman J, Lesk A. Amyloid fibers are water-filled nanotubes. Proc Natl Acad Sci USA 2002; 99: 5591-5595. doi:10.1073/pnas.042681399
[32] Touw W G, Joosten R P, Vriend G. Detection of trans-cis flips and peptide-plane flips in protein structures. Acta Crystallogr D Biol Crystallogr 2015; 71: 1604-1614. doi:10.1107/S1399004715008263
[33] Ellisdon A M, Thomas B, Bottomley S P. The two-stage pathway of ataxin-3 fibrillogenesis involves a polyglutamine-independent step. J Biol Chem 2006; 281: 16888-16896. doi:10.1074/jbc.M601470200 A. B. Bransgrove and L. A. Anderson Australian Journal of Chemistry 598
[34] Marchut A J, Hall C K. Side-chain interactions determine amyloid formation by model polyglutamine peptides in molecular dynamics simulations. Biophys J 2006; 90: 4574-4584. doi:10.1529/biophysj.105.079269
[35] Plumley J A, Dannenberg J J. The importance of hydrogen bonding between the glutamine side chains to the formation of amyloid VQIVYK parallel β-sheets: an ONIOM DFT/AM1 study. J Am Chem Soc 2010; 132: 1758-1759. doi:10.1021/ja909690a
[36] Kobko N, Paraskevas L, del Rio E, Dannenberg J J. Cooperativity in amide hydrogen bonding chains: implications for protein-folding models. J Am Chem Soc 2001; 123: 4348-4349. doi:10.1021/ja004271l
[37] Kobko N, Dannenberg J J. Cooperativity in amide hydrogen bonding chains. Relation between energy, position, and H-bond chain length in peptide and protein folding models. J Phys Chem A 2003; 107: 10389-10395. doi:10.1021/jp0365209
[38] Venkatraman P, Wetzel R, Tanaka M, Nukina N, Goldberg A L. Eukaryotic proteasomes cannot digest polyglutamine sequences and release them during degradation of polyglutamine-containing proteins. Mol Cell 2004; 14:95-104. doi:10.1016/S1097-2765(04)00151-0
[39] Nijholt D A T, De Kimpe L, Elfrink H L, Hoozemns J J M, Scheper W. Removing protein aggregates: the role of proteolysis in neurodegeneration. Curr Med Chem 2011; 18: 2459-2476. doi:10.2174/092986711795843236
[40] Lin L-N, Brandts J F. Role of cis-trans isomerism of the peptide bond in protease specificity: kinetic studies on small proline containing peptides and on polyproline. Biochemistry 1979; 18: 5037-5042. doi:10.1021/bi00590a002
[41] Zhang Y, Wölfle T, Rospert S. Interaction of nascent chains with the ribosomal tunnel proteins Rp14, Rpl17, and Rpl39 of Saccharomyces cerevisiae. J Biol Chem 2013; 288: 33697-33707. doi:10.1074/jbc.M113.508283
[42] Komar A A. From alpha to beta-a co-translational way to fold? Cell Cycle 2022; 21: 1663-1666. doi:10.1080/15384101.2022.2062186
[43] Syroegin E A, Aleksandrova E V, Polikanov Y S. Insights into the ribosome function from the structures of non-arrested ribosome-nascent chain complexes. Nat Chem 2023; 15: 143-153. doi:10.1038/s41557-022-01073-1
[44] Orsi R H, Bowen B M, Wiedmann M. Homopolymeric tracts represent a general regulatory mechanism in prokaryotes. BMC Genomics 2010; 11: 102. doi:10.1186/1471-2164-11-102
[45] Haran T E, Mohanty U. The unique structure of A-tracts and intrinsic DNA bending. Q Rev Biophys 2009; 42: 41-81. doi:10.1017/S0033583509004752
[46] Brodsky S, Jana T, Barkai N. Order through disorder: the role of intrinsically disordered regions in transcription factor binding specificity. Curr Opin Struct Biol 2021; 71: 110-115. doi:10.1016/j.sbi.2021.06.011
[47] Gemayel R, Chavali S, Pougach K, Legendre M, Zhu B, Boeynaems S, van der Zande E, Gevaert K, Rousseau F, Schymkowitz J, Babu M M, Verstrepen K J. Variable glutamine-rich repeats modulate transcription factor activity. Mol Cell 2015; 59: 615-627. doi:10.1016/j.molcel.2015.07.003
[48] Atanesyan L, Günther V, Dichtl B, Georgiev O, Schaffner W. Polyglutamine tracts as modulators of transcriptional activation from yeast to mammals. Biol Chem 2012; 393: 63-70. doi:10.1515/BC-2011-252
[49] Tang W., Zhou H., Li W. Silver and cyanine staining of oligonucleotides in polyacrylamide gel. PLos One 2015: 10 e0144422. doi: 10.1371/journal.pone. 01444222
[50] Patel A., et al. ATP as a biological hydrotrope. 2017; 356 753-756.doi:10.1126/science.aaf6846
[51] Haye M; Peuchem E; Douichin; Week D. elife. 2018; 7:e35224. doi.org/10.7554/elife.35224
[52] Takaine M; Imamura H; Yoshida S. elife 2022; doi.org/10.7554/elife. 67659

1. A method for binding a nucleotide to a polypeptide comprising combining the nucleotide with soluble or aggregated polypeptide. The aggregates may take the form of soluble aggregates or insoluble aggregates.

2. The method of claim 1 wherein the polypeptide is a homopolymer.

3. The method of claim 2 wherein the homopolymer is a polyglutamine repeat.

4. The method of claim 3 wherein the polyglutamine repeat can be any length up to 100 residues.

5. The method of claim 4 wherein the polyglutamine repeat sequence is comprised of 15 glutamine residues with N-terminal acetylation and C- terminal amidation (polyQ15).

6. The method of claim 5 wherein the C-terminal amidation is replaced by 2 lysine residues to augment solubility (polyQ15kk).

7. The method of claim 5 wherein the poly(Q15) dissolves in pure water, at the rate of 1 mg/ml to form visual aggregates, usually within 24 to 48 hours.

8. The method of claim 6 wherein the poly(Q15kk) dissolves in pure water, at the rate of 1 mg/ml but forms aggregates usually after 7 days.

9. The method of claim 5 wherein the addition of buffer causes immediate visually recognisable aggregation.

10. The method of claim 3 wherein the polyglutamine side chain amide groups are linked by cooperatively enhanced H-bonds to form a linear chain extending for the length of the polymer.

11. The method of claim 4 wherein the cooperative energy of formation of the linear amide chain is sufficient to overcome the energy barrier preventing the flip of trans amide to the cis isomer.

12. The method of claim 4 wherein the polyglutamine repeat backbone is in the all cis conformation.

13. The method of claim 2 wherein the homopolymer is an all cis conformation of certain convenient amino acids.

14. The method of claim 9 wherein the linear H-bonded amide chain functions as a scaffold for H-bond cross-linking to other identical linear H-bonded amide chains giving rise to dimers, trimers, or more, forming sheets and utilising all four potential H bonds of each side chain amide.

15. The method of claim 1 wherein the nucleotide is adenosine mono, di, or triphosphate.

16. The method of claim 1 wherein the nucleotide is a polynucleotide.

17. The method of claim 15 wherein the polynucleotide is polyadenosine monophosphate. Referred to elsewhere in this application as polyadenine (polyA).

18. The method of claim 16 wherein the poly(A) repeat sequence is comprised of in excess of 100 adenosine monophosphate residues.

19. The method of claim 18 wherein the poly(A) repeat sequence is comprised of 20 adenosine monophosphate residues

20. The method of claim 17 wherein the binding of poly(A) to the poly(Q15), (claim 5) separates and isolates the monomer form of poly(Q15) as a soluble complex from oligomers or aggregates.

21. The method of claim 20 wherein the poly(A) can be any length up to 100 residues 22. The method of claim 15 wherein Adenosine triphosphate (ATP) is combined with poly(Q15) (claim 5) and will solubilize the peptide when in amounts exceeding 10 mM.

23. The method of claim 22 wherein an amount of ATP in excess of 10 mmol/L will solubilise the peptide aggregates.

24. The method of claim 20 wherein the binding between the two polymers can be monitored using acrylamide gel electrophoresis.

25. The method of claim 19 wherein the binding between poly(Q15) and ATP can be monitored with TLC

26. The method of claim 24 wherein the undissolved aggregates on TLC persist at the origin and soluble monomers migrate.

27. The method of claim 15 wherein H-bond breakers are included in the mix with nucleotides as a secondary function to aid aggregate breakdown.

28. The method of claim 25 where the H-bond breaker is formamide.

29. A method is describe wherein the H bonded amides of the glutamine side chains provide a scaffold onto which successive monomers may attach by H bond cross-linkage to expand the sheet of multiple polyQ monomers.

30. The method of claim 29 wherein the repeated attachment of polyQ monomers to the scaffold, but out of register, provides a means to generate fibre growth.
